(19) United States
(12) Patent Application Publication
(10) Pub. No.: US 2024/0293329 A1
(43) Pub. Date: Sep. 5, 2024

(54) PHARMACEUTICALLY STABLE SOFT CAPSULE COMPRISING TWO OR MORE DIFFERENT COMPOSITIONS

(71) Applicant: COSMAXPHARMA CO.,LTD., Chungchcheongbuk-do (KR)

(72) Inventors: Myounghoon PARK, Uiwang-si Gyeonggi-do (KR); Sohee YOON, Seongnam-si Gyeonggi-do (KR); Subong HONG, Seoul (KR); Jihye CHOI, Seoul (KR)

(73) Assignee: COSMAXPHARMA CO.,LTD., Cheongju-si Chungcheongbuk-do (KR)

(21) Appl. No.: 18/268,091

(22) PCT Filed: Dec. 20, 2021
(86) PCT No.: PCT/KR2021/019367

§ 371 Date: Oct. 27, 2023

(30) Foreign Application Priority Data
Dec. 18, 2020 (KR) 10-2020-0178666

(51) Int. Cl. A61K 9/48; A61J 3/07; A61K 45/06

(52) U.S. Cl. CPC A61K 9/4808; A61J 3/07; A61K 9/4825; A61K 9/4866; A61K 9/4875; A61K 45/06

(57) ABSTRACT

The present disclosure relates to a pharmaceutically stable soft capsule formulation containing two or more different compositions, wherein each single ingredient can be formulated as a composite formulation, and a formulation with improved stability can be provided by minimizing a reaction between two or more different compositions.

### TECHNICAL FIELD

The present disclosure relates to a pharmaceutically stable soft capsule formulation containing two or more different types of compositions.

### BACKGROUND ART

Conventional soft capsules are known to be filled with oily raw materials, a liquid phase in which drugs and active ingredients are dissolved, or a suspension phase in which a solid or a dry solid are dispersed in liquid. Soft capsules are formulations with special advantages not only for substances that require complete protection from air and light, but also for refined fish oil or tocopherol, in which active ingredients are oily.

Such soft capsules have been widely known for decades, and have been used in health functional foods, pharmaceuticals, cosmetics, and the like. Soft capsules generally contain an outer gelatin shell formulation made of a gelatin, a plasticizer, and water, and filling substances inside a capsule. The filling substances may be selected from a variety of compatible materials that are not reactive with a gelatin shell formulation. Soft capsules may be used in various sizes in a circle, oval, or tube shape, and may be prepared in various colors by adding a pigment in the preparation of a gelatin shell formulation. On the other hand, as development of sustained-release formulations or complexes has recently become active to increase the convenience of taking solid formulations, various sustained-release technologies and products such as multi-layer tablets are being distributed. Even in the case of soft capsules, technologies for containing tablets or capsules in soft capsules are emerging to compensate for the existing disadvantages. However, development of a soft capsule formulation, in which two or more different compositions are filled in a single soft capsule without preparing separate granules, is necessary.

### DISCLOSURE

### Technical Problem

An aspect provides a capsule formulation in which a first composition including a first pharmaceutical active ingredient and a second composition including a second pharmaceutical active ingredient are present in separate phases from each other.

Another aspect provides a capsule formulation including: a continuous first phase of a first liquid phase or a first suspension including a base in which a first pharmaceutical active ingredient is dissolved or dispersed; and a continuous second phase of a second liquid phase or a second suspension including a base in which a second pharmaceutical active ingredient is dissolved or dispersed.

Another aspect provides a capsule molding device including: a first receiving chamber including a base, in which a first pharmaceutical active ingredient is dissolved or dispersed, and receiving a first liquid phase or a first suspension; a second receiving chamber including a base, in which a second pharmaceutical active ingredient is dissolved or dispersed, and receiving a second liquid phase or a second suspension; a sheet forming unit configured to form a sheet using a gelatin shell formulation base solution; a capsule molding unit including a pair of die rolls arranged adjacent to the sheet forming unit to be supplied with a sheet formed from the sheet forming unit and being configured to encapsulate the sheet; and an injection unit arranged adjacent to the outer periphery of the pair of die rolls and including an injection segment for injecting the first liquid phase or the first suspension supplied from the first receiving chamber and the second liquid phase or the second suspension supplied from the second receiving chamber into the encapsulated sheet, wherein the first liquid phase or the first suspension and the second liquid phase or the second suspension are present as continuous phases that are separate from each other within the capsule formulation, and the injection segment includes: a first injection hole connected to the first receiving chamber; a second injection hole connected to the second receiving chamber; a first ejection hole being in communication with the first injection hole; and a second ejection hole being in communication with the second injection hole.

### Technical Solution

An aspect provides a pharmaceutically stable soft capsule formulation containing two or more different compositions.

In an embodiment, the soft capsule formulation may be a capsule formulation including: a continuous first phase of a first liquid phase or a first suspension including a base in which a first pharmaceutical active ingredient is dissolved or dispersed; and a continuous second phase of a second liquid phase or a second suspension including a base in which a second pharmaceutical active ingredient is dissolved or dispersed.

In an embodiment, the capsule formulation may be selected from the group consisting of the following cases:
(1) the first liquid phase or the first suspension contains a water-soluble base, and the second liquid phase or the second suspension contains a fat-soluble base, or vice versa;
(2) the first liquid phase or the first suspension contains a fat-soluble base, and the second liquid phase or the second suspension contains a fat-soluble base; and
(3) the first suspension contains a water-soluble base, and the second suspension contains a water-soluble base.

In an embodiment, the first phase and the second phase may be present as separate phases from each other. The capsule formulation according to an embodiment consists of: the continuous first phase loaded with the first pharmaceutical active ingredient; and the continuous second phase loaded with the second pharmaceutical active ingredient, wherein the continuous first phase and the continuous second phase may be separated without a separate surfactant or the presence of a physical layer. Such phase separation is distinguished from separation in which a discontinuous phase is dispersed within another phase as in a general water-in-oil or oil-in-water type, and refers to separation in which two phases do not overlap regions of one another (or in which one phase does not invade a region of another phase, or in which one phase does not overlap a region of another phase).

Therefore, the first phase and the second phase of the capsule formulation according to an embodiment may be present as continuous phases, and may not overlap with an existence region of phases of each other.

In one or more embodiments, one of the continuous first phase and the continuous second phase may be present as two or multiple continuous phases that are separated from each other through the other phase.

In the capsule formulation, the first liquid phase or suspension and the second liquid phase or suspension may be ejected from two ejection holes of two separate receiving chambers of a capsule molding device, and may simultaneously be filled in a capsule formulation to be formulated in one capsule. Also, the first liquid phase or the first suspension is not miscible with the second liquid phase or the second suspension, since ingredients of the base of the first liquid phase or the first suspension are different from ingredients of the base of the second liquid phase or the second suspension, and physical properties of the first liquid phase or the first suspension are different from physical properties of the second liquid phase or the second suspension. In this regard, the continuous first phase and the continuous second phase in the capsule formulation may be present as separate phases from each other without an additional surfactant or the presence of a physical layer.

In the present specification, the “capsule formulation” may be prepared by filling a capsule with health functional foods, pharmaceuticals, or drugs, or by shaping the same as a capsule film. Therefore, the capsule formulation according to an embodiment may further include a pharmaceutical or nutraceutical soft capsule gelatin shell layer (gelatin shell base). The capsule formulation may be a formulation in which health functional foods, pharmaceuticals, or drugs are filled in a gelatin shell layer, and the gelatin shell layer may consist of, for example, one or more ingredients selected from the group consisting of starch, arabic gum, tragacanth gum, karaya gum, ghatti gum, guar gum, locust bean gum, tara gum, konjac gum, algin, agar, carrageenan, pullulan, pectin, gellan, mannan, gelatin, xanthan gum, and a mixture thereof. The capsule formulation may further include a plasticizer suitable for use in gelatin shells. The plasticizer may include one or more ingredients selected from the group consisting of glycerin, ethyl phthalate, triethyl citrate, dibutyl sebacate, polyethylene glycol, triacetin, tributyl citrate, propylene glycol, and a mixture thereof.

Also, in the preparation of the gelatin shell layer, other additives may be included within a range that does not impair the appearance stability, disintegration speed, and formulation homogeneity of the soft capsule. For example, the gelatin shell layer, i.e., the base of the soft capsule, may be prepared by adding glycerin, sugar alcohol, and/or purified water to gelatin, and gelatin shell layer may further include a colorant, a flavoring agent, a preservative, or a gelatin shell base to improve the appearance of the soft capsule.

In the capsule formulation, without an additional surfactant or the presence of a physical layer, the first liquid phase or the first suspension; and the second liquid phase or the second suspension may be present as separate phases from each other. In general, in the case of a composite formulation of a soft capsule dosage, a second drug including an additional gelatin shell layer may be incorporated into a capsule formulation containing a first drug; or a liquid substance may be contained in a first drug, and a second drug may be included in the form of a tablet or the like inside a capsule. In this regard, an inappropriate reaction between the first drug and the second drug may be prevented. However, in the capsule formulation according to an aspect, despite not being in a state where the second liquid phase or the second suspension is incorporated into the first liquid phase or the first suspension or a state where the first liquid phase or the first suspension is incorporated into the second liquid phase or the second suspension, the reactivity between active ingredients may be minimized by phase separation. In an embodiment, by using a capsule formulation containing two or more different active ingredients having different efficacy or release characteristics of the same drug, the contents of the active ingredients in the capsule formulation did not change during a long storage period, and thus it was confirmed that the capsule formulation had excellent stability. Therefore, the capsule formulation according to an aspect may be dissolved and released within an appropriate time range, resulting in high bioavailability. Accordingly, the drug efficacy may be maximized.

The water-soluble base may be any one selected from the group consisting of polyethylene glycol, propylene glycol, polymethyl acrylate, polyethylene oxide, saturated polyglycorized glyceride (Gelucire), glycerol monostearate, carbohydrate, cellulose, polyvinyl alcohol, polyacrylic acid, propylene carbonate, diethylene glycol monoethyl ether (Transcutol), triacetin, concentrated glycerin, glycerol monocaprylocaprate, tetraglycol, and a combination thereof.

The fat-soluble base may be: plant oil, such as soybean oil, coconut oil, sunflower seed oil, sesame oil, perilla oil, palm oil, olive oil, castor oil, or polyoxyl hydrogenated castor oil; animal oil, such as squalane, refined fish oil, and the like; mineral oil, such as vaseline, liquid paraffin, paraffin, ozokeraite, ceresin, microcrystalline wax, and the like; medium chain fatty acid triglyceride; and the like.

In the present specification, the term “pharmaceutical active ingredient” refers to a physiologically active substance, and may refer to a substance administered for the purpose of improving, preventing, or treating a disease condition or abnormal condition of an individual, or symptoms related thereto, and may include ingredients that can be included in medicines, foods, health functional foods, cosmetics, beauty products, quasi-drugs, medical devices, and the like. In addition, the first pharmaceutical active ingredient and the second pharmaceutical active ingredient may be the same or different from each other.

The first pharmaceutical active ingredient may be selected from the group consisting of a non-steroidal anti-inflammatory drug (NSAID), a flue medicine ingredient, a vitamin, and a statin-based drug. In the present specification, the “non-steroidal anti-inflammatory drug (NSAID)” collectively refers to a substance that does not have a steroid structure and inhibits COX, and may include the following ingredients. The following ingredients may be a free acid or a salt thereof of the active ingredient itself, or may be a free acid or a salt thereof of an isomer of the active ingredient.
salicylates: aspirin, dipulunisal, salicylic acid or a derivative thereof, salsalate, etc
propionic acid derivates: ibuprofen, fenoprofen, flurbiprofen, benoxaprofen, fenbufen, dexibuprofen, ketoprofen, oxaprozin, naproxen, dexketoprofen, loxoprofen, indoprofen, pirprofen, carprofen, pranoprofen, miroprofen, tioxapropen, suprofen, alminoprofen
acetic acid derivatives: indomethacin, sulindac, ketorolac, aceclofenac, tometin, etodolac, dilofenac, nabumetone
oxycam derivatives: piroxicam, tenoxicam, lornoxicam, phenylbutazone, meloxicam, droxicam, isoxicam
anthranilic acid derivatives: mefenamic acid, meclofenamic acid, flufenamic acid, tolfenamic acid
nimesulide, celecoxib, rofecoxib, valdecoxib, lumiracoxib.

In the present specification, the “influenza drug ingredient” refers to an ingredient used as a cough expectorant, a decongestant, or the like, not only having an effect of relieving fever, toothache, neuralgia, muscular pain, or the like caused by influenza, but also having an analgesic effect, an anti-inflammatory effect, an anti-pyretic effect, or the like. The flue drug ingredient may include, for example, choline salicylate, salicylamide, aspirin, ethenzamide, acetaminophen, ibuprofen, dextromethorphan hydrobromide, noscapine. HCl, trimethoquinol. HCl, guaifenesin, d-chlorpheniramine maleate, carbetapentane citrate, tipepidine citrate, cloperastine HCl, cloperastine fendizoate, tipepidine hibenzate, DL-methylephedrine HCl, ephedrine HCl, phenylephrine HCl, pseudoephedrine HCl, phenylpropanolamine, diphexamide, phenylaminopropanol HCl, oxymetazoline, xylometazoline, tripelenamine hydrochloride, triprolidine hydrochloride, diphenhydramine hydrochloride, alimemazine tartrate, diphenylpyraline hydrochloride, brompheniramine maleate, doxylamine succinate, pheniramine maleate, mepiramine maleate, diphenhydramine tannate, diphenhydramine citrate, alloclamide HCl, noscapine hydrochloride, noscapine, phenylephrine HCl, potassium guaiacolsulfonate, serratiopeptidase, semi-alkaline protease, caffeine, caffeine and sodium benzoate, or the like.

Also, the first pharmaceutical active ingredient may include an HMG-COA reductase inhibitor, and specifically may be a statin-based drug. In the specification, the “statin-based drug” can inhibit an HMG-Coa reductase, which regulates cholesterol production yields in the body, so that cholesterol can be reduced by slowing the cholesterol production or by increasing the ability of the liver to remove LDL cholesterol already present in the blood. The statin-based drug may include, for example, atorvastatin, rosuvastatin, lovastatin, simvastatin, pravastatin, fluvastatin, cerivastatin, pitavastatin, and a pharmaceutically acceptable salt thereof.

The second pharmaceutical active ingredient may be in a liquid phase, and specifically may be in an oil phase. For example, the second pharmaceutical active ingredient may be selected from the group consisting of an omega-3 fatty acid or alkyl ester thereof, an antacid, and a vitamin. The omega-3 fatty acid or alkyl ester thereof may serve to lower levels of serum triglyceride (TG), lower systolic and diastolic blood pressures and pulse rates, and lower the activity of the blood coagulation factor VII phospholipid complex, with few side effects on the human body. In the present specification, the “omega-3 fatty acid” includes all of those referred to as to ω-3 unsaturated fatty acid, ω-3 highly unsaturated fatty acid, polyunsaturated fatty acid (PUFA), and examples thereof includes docosahexaenoic acid (DHA), eicosapentaenoic acid (EPA), arachidonic acid (ARA), docosapentaenoic acid, α-linolenic acid, and a mixture thereof. In an embodiment, the omega-3 fatty acid alkyl ester may be C1-C3 alkyl ester, and may be ethyl ester, such as ethyl ester of DHA or ethyl ester of EPA.

In the present specification, the “antacid” refers to a compound capable of relieving heartburn feeling (or pyrosis) typical in acid hypersecretion, and it acts both directly on hyperacidity and gastroesophageal reflux, i.e., by buffering the pH of the gastric mucosa, or indirectly by inhibiting acid secretion from the stomach. The antacid may be: for example, magaldrate or sucralfate; citrate, such as sodium citrate or potassium citrate; magnesium oxide; magnesium hydroxide; magnesium carbonate; magnesium silicate, such as magnesium trisilicate; dihydroxyaluminum aminoacetate; aluminium oxide; aluminium hydroxide; bicarbonate, such as sodium bicarbonate; carbonate, such as calcium carbonate; alginic acid; sodium alginate; calcium phosphate; hydrotalcite; aluminum glycinate; galactan sulfate; myrtecaine; or the like.

In the present specification, the “vitamin” refers to a substance that plays an essential role in health by helping the individual body function properly, and plays a role related to body reactions. The vitamin may be classified as a water-soluble vitamin and a fat-soluble vitamin, according to the way of absorption and storage. In an embodiment, the vitamin may be a fat-soluble vitamin or a water-soluble vitamin. The fat-soluble vitamin may be, for example, vitamin D2 or D3, vitamin E or E-acetate, vitamin A, vitamin K1, vitamin K2, or a provitamin or prodrug of vitamins K1 and K2. Also, the water-soluble vitamin may be, for example, vitamins B1, B2, B6, B12, and C, folic acid, biotin, nicotinic acid amide, and the like.

The first pharmaceutical active ingredient or the second pharmaceutical active ingredient may be acetaminophen, tramadol hydrochloride, aceclofenac, naproxen, esomeprazole magnesium trihydrate, cetirizine hydrochloride, pseudoephedrine hydrochloride, ebastine, cilostazol, glimepiride, metformin hydrochloride, sitagliptin phosphate hydrate, galantamine hydrobromide, saxagliptin monohydrate, amlodipine besylate, valsartan, telmisartan, hydrochlorothiazide, olmesartan medoxomil, rosuvastatin calcium, naproxen sodium, sumatriptan, pramipexole dihydrochloride monohydrate, clonidine HCl, nicardipine HCl, doxazosin mesylate, indapamide, felodipine, tolterodine l-tartrate, ritodrine HCl, tamsurosin HCl, nifedipine, isosorbide mononitrate (or isosorbide dinitrate), nisoldipine, venlafaxine HCl, trazodone HCl, paroxetine hydrochloride hydrate, roxatidine acetate HCl, metoclopramide hydrochloride hydrate, salbutamol sulphate, orphenadrine citrate, chlormadinone acetate, oxybutynin hydrochloride, and the like.

In an embodiment, the capsule formulation may be a mixture of an anti-inflammatory analgesic active ingredient and an antacid. By mixing the anti-inflammatory analgesic active ingredient and the antacid, gastrointestinal discomfort that may occur when taking the anti-inflammatory analgesic may be relieved.

In one or more embodiments, the capsule formulation may be a mixture of a flue drug ingredient and a vitamin. By mixing the flue drug ingredient and the vitamin, an excellent effect for recovering from flu symptoms may be exhibited.

In one or more embodiments, the capsule formulation may be a mixture of a statin-based drug and omega-3 fatty acid or alkyl ester thereof. By mixing the statin-based drug and the omega-3 fatty acid or alkyl ester thereof, not only can a synergistic effect of the statin-based drug and the omega-3 be expected, but also the discomfort of patients who have to take the two drugs can be relieved, thereby increasing medication compliance.

As described above, the capsule formulation according to an aspect can formulate a composite formulation of each single agent, and can minimize the reactivity between the active ingredients due to the phase separation without an additional surfactant or the presence of a physical layer. In this regard, two or more active ingredients can be independently delivered to desired sites without loss of the active ingredients.

Another aspect provides a capsule molding device.

A multi(double)-fill shaping device according to an embodiment may include:
a first receiving chamber including a base in which a first pharmaceutical active ingredient is dissolved or dispersed, and receiving a first liquid phase or a first suspension;
a second receiving chamber including a base in which a second pharmaceutical active ingredient is dissolved or dispersed, and receiving a second liquid phase or a second suspension;
a sheet forming unit configured to form a sheet using a gelatin shell formulation base solution;
a capsule molding unit including a pair of die rolls arranged adjacent to the sheet forming unit to be supplied with a sheet formed from the sheet forming unit and being configured to encapsulate the sheet; and
an injection unit arranged adjacent to the outer periphery of the pair of die rolls and including an injection segment for injecting the first liquid phase or the first suspension supplied from the first receiving chamber and the second liquid phase or the second suspension supplied from the second receiving chamber into the encapsulated sheet,
wherein the first liquid phase or the first suspension and the second liquid phase or the suspension may be present as continuous phases that are separated from each other within the capsule formulation, and
the injection segment may include: a first injection hole connected to the first receiving chamber; a second injection hole connected to the second receiving chamber; a first ejection hole being in communication with the first injection hole; and a second ejection hole being in communication with the second injection hole. Accordingly, effects that the two phases do not mix with each other and can be formulated in one capsule may be exhibited.

In an embodiment, the capsule molding device may further include at least one (e.g., 1, 2, or 3) receiving chamber. The at least one receiving chamber further included may operate in the same way as the first receiving chamber or the second receiving chamber in the capsule molding device, and in this regard, a capsule having at least 3, 4, or 5 phases may be formulated.

In an embodiment, the capsule formulation may be prepared by using the capsule molding device.

### Advantageous Effects of Disclosure

In the capsule formulation according to an aspect, individual single ingredients can be formulated as a composite formulation, and a formulation having improved stability by minimizing a reaction between two or more different types of compositions can be provided.

### DETAILED DESCRIPTION OF THE DRAWINGS

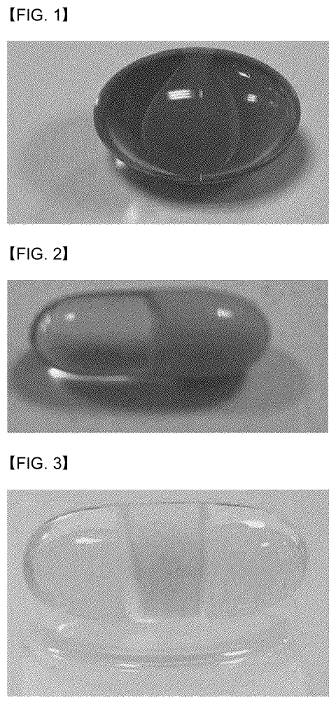

FIG. 1 is a photograph of a soft capsule of Example 1 according to an embodiment.

FIG. 2 is a photograph of a soft capsule of Example 2 according to an embodiment.

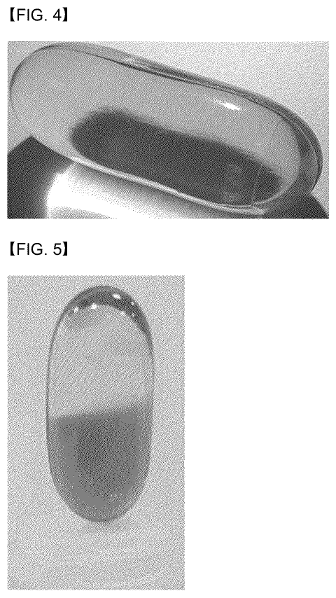

FIG. 3 is a photograph of a soft capsule of Example 2 according to an embodiment.

FIG. 4 is a photograph of a soft capsule of Example 3 according to an embodiment.

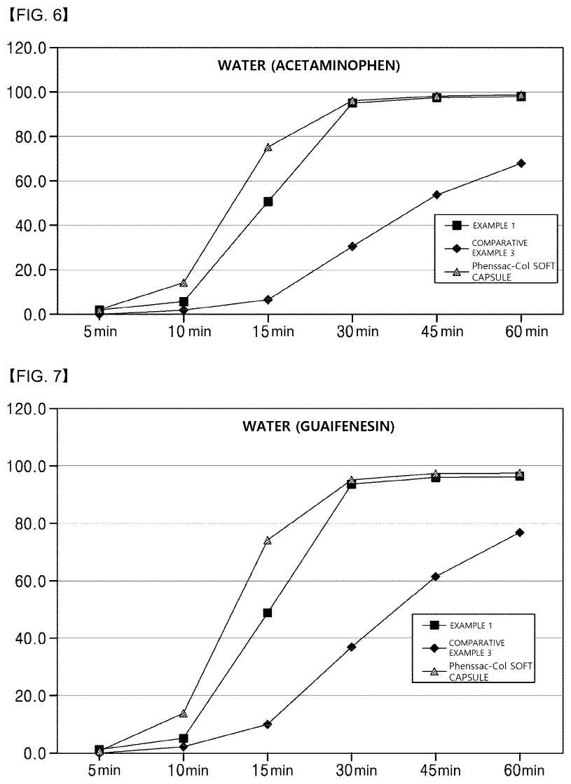

FIG. 5 is a photograph of a soft capsule of Example 4 according to an embodiment.

FIG. 6 is a graph comparing the average dissolution rates of the active ingredient (acetaminophen) of Example 1, Comparative Example 3, and Phenssac-Col soft capsule.

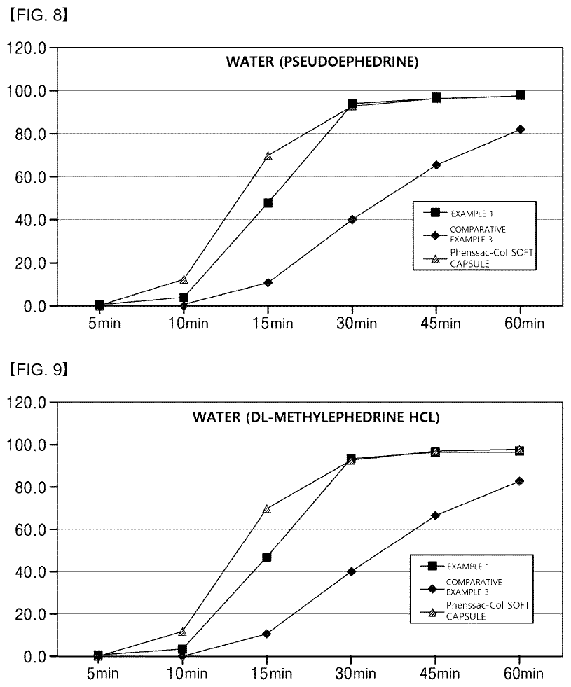

FIG. 7 is a graph comparing the average dissolution rates of the active ingredient (guaifenesin) of Example 1, Comparative Example 3, and Phenssac-Col soft capsule.

FIG. 8 is a graph comparing the average dissolution rates of the active ingredient (pseudoephedrine) of Example 1, Comparative Example 3, and Phenssac-Col soft capsule.

FIG. 9 is a graph comparing the average dissolution rates of the active ingredient (DL-methylephedrine HCl) of Example 1, Comparative Example 3, and Phenssac-Col soft capsule.

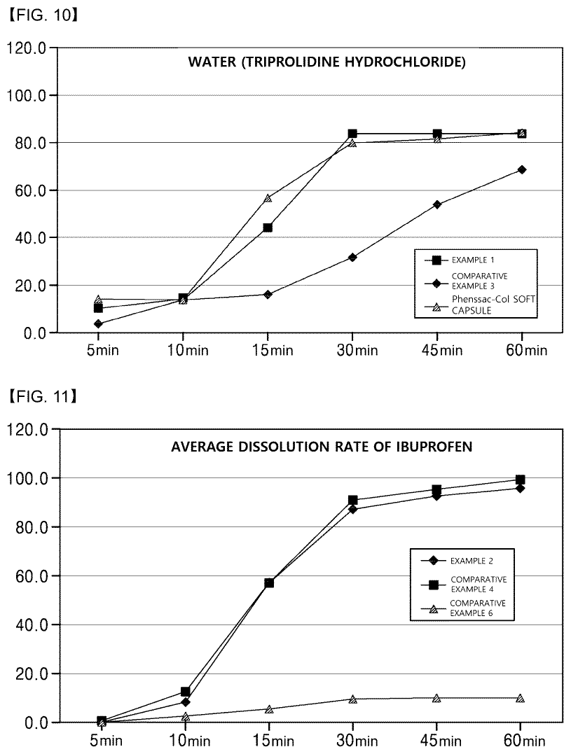

FIG. 10 is a graph comparing the average dissolution rates of the active ingredient (triprolidine hydrochloride) of Example 1, Comparative Example 3, and Phenssac-Col soft capsule.

FIG. 11 is a graph comparing the average dissolution rates of the active ingredient (ibuprofen) of Example 2 and Comparative Examples 4 and 6.

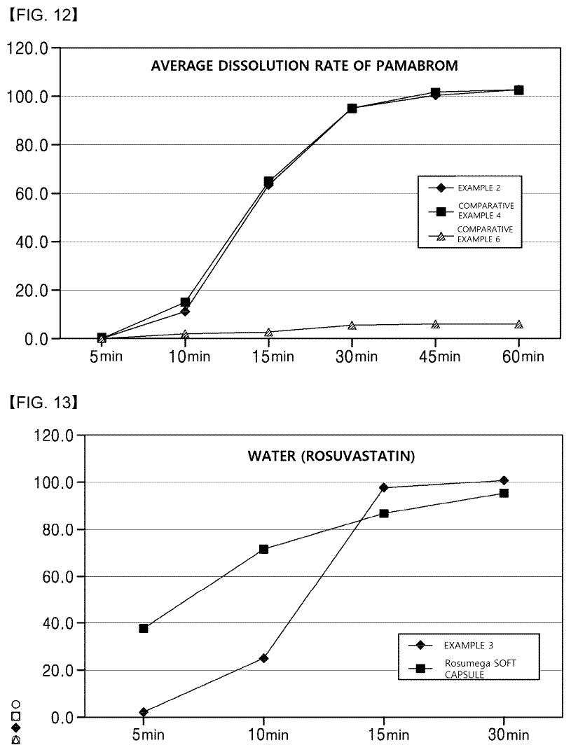

FIG. 12 is a graph comparing the average dissolution rates of the active ingredient (pamabrom) of Example 2 and Comparative Examples 4 and 6.

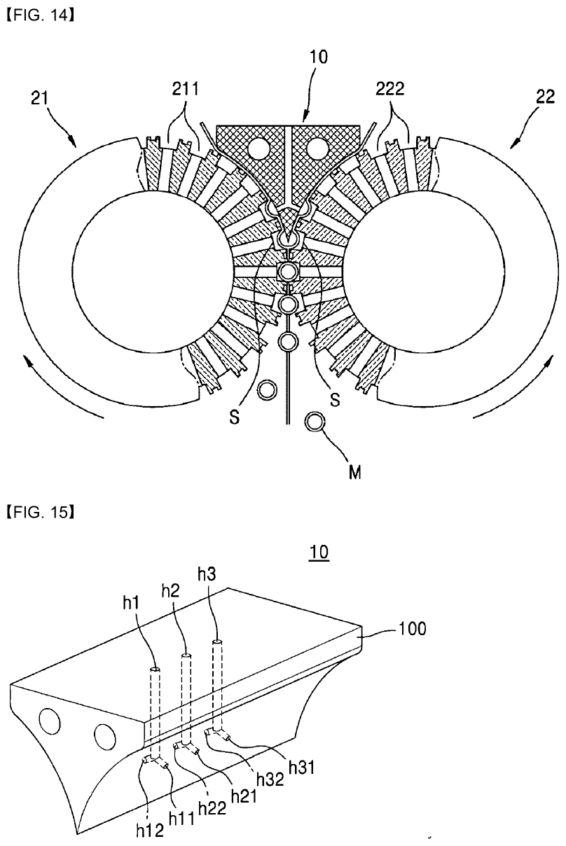

FIG. 13 is a graph comparing the average dissolution rates of the active ingredient (rosuvastatin) of Example 3 and Rosumega soft capsule.

FIG. 14 is a diagram schematically illustrating a portion of a capsule molding device according to an embodiment of the present disclosure.

FIG. 15 is a diagram for explaining an injection unit of FIG. 14.

### MODE FOR INVENTION

Hereinafter, preferable Examples are presented to help understanding of the present disclosure. However, the following examples are only presented for easier understanding of the present disclosure, and the contents of the present disclosure are not limited by the following examples.

FIGS. 2, 4, and 5 are photographs of soft capsules of Examples 2, 3, and 4, respectively, according to an embodiment. As shown in FIGS. 2, 4, and 5, a soft capsule according to an embodiment consists of a continuous first phase loaded with a first pharmaceutical active ingredient and a continuous second phase loaded with a second pharmaceutical active ingredient, wherein the continuous first phase and the continuous second phase are separated without an additional surfactant or the presence of a physical layer. Such phase separation is distinguished from separation in which a discontinuous phase is dispersed within another phase as in a general water-in-oil or oil-in-water type, and refers to separation in which two phases do not overlap regions of one another (or in which one phase does not invade a region of another phase, or in which one phase does not overlap a region of another phase).

FIGS. 1 and 3 are photographs of soft capsules of Examples 1 and 2, respectively, according to an embodiment. As shown in FIGS. 1 and 3, one of the continuous first phase and the continuous second phase may be present as two or multiple phases that are separated from each other through the other phase. That is, the continuous second phase may be present as two or multiple phases separated from each other through the continuous first phase (or vice versa). Such phase separation also refers that multiple phases are separated without overlapping regions of each other (or one phase does not invade a region of the other phase, or one phase does not overlap a region of the other phase.

### EXAMPLES

### Example 1. Soft Capsule Containing Aqueous Solution and Fat-Soluble Suspension

A soft capsule containing an aqueous solution and a fat-soluble suspension according to ingredients and contents shown in Table 1 below was prepared. Unless otherwise indicated in the present specification, the content is expressed in wt %. In detail, polyethylene glycol 400 as a water-soluble base and propylene glycol as a dissolution auxiliary agent were added to purified water, and mixed at a speed of 150 rpm for 10 minutes. Afterwards, povidone as a dissolution auxiliary agent was added to the mixture, and mixed at about 60° C. at a speed of 400 rpm, followed by complete dissolution to prepare a water-soluble base (first base). The first active ingredients was added one by one to the prepared base, and then dissolved while continuously mixing at a speed of about 400 rpm at 60° C. until the active ingredients added therein were completely dissolved. Next, the mixture was filtered through 200 mesh and cooled, and air bubbles were removed therefrom to prepare a filling composition for a water-soluble soft capsule (first composition).

Hard fat and yellow wax as suspending agents were added to soybean oil as a fat-soluble base, and mixed at a speed of 600 rpm at about 55° C. until the ingredients added therein were completely dissolved to prepare a fat-soluble base (second base). To the prepared base, lecithin as a wetting agent, tocopherol acetate as an antioxidant, and a second active ingredient were added, and mixed at the same speed for about 15 minutes until being blended homogeneously. Afterwards, the blended mixture was milled by using a colloid mill, filtered through 80 mesh, and cooled, and air bubbles were removed therefrom to prepare a filling composition for a fat-soluble soft capsule (second composition).

These two types of filling compositions for a soft capsule thus prepared were mixed with gelatin as a gelatin shell base for a soft capsule and sorbitol sorbitan solution and water as plasticizers to form gel mass. Then, a soft capsule was prepared by using a multi(double)-fill shaping device using the gel mass and a soft capsule gelatin shell formulation formed in a thin and wide ribbon form.

TABLE 1

| | Blending | | |
|---|---|---|---|
| Division | purpose | Ingredient | Content (wt %) |
| Filling | First active | Acetaminophen | 16.07 |
| composition for | ingredient | Guaifenesin | 1.86 |
| water-soluble | | Pseudoephedrinehydro- | 1.34 |
| soft capsule | | chloride | |
| | | DL-methylephedrine HCl | 1.12 |
| | | Triprolidine | 0.06 |
| | | hydrochloridehydrate | |
| | Additive | Polyethylene glycol 400 | 30.64 |
| | | Purified water | 4.11 |
| | | Propylene glycol | 2.31 |
| | | Povidone | 2.31 |
| Filling | Second | Ascorbic acid | 4.46 |
| composition for | active | Riboflavin | 0.18 |
| fat-soluble | ingredient | Thiamine nitrate | 0.37 |
| soft capsule | Additive | Soybean oil | 7.22 |
| | | Hard fat | 1.98 |
| | | Yellow lead | 0.66 |
| | | Lecithin | 0.27 |
| | | Tocopherol acetate | 0.03 |
| Gelatin shell | Gelatin | Gelatin | 15.88 |
| formulation of | shell | Sorbitol sorbitan | 9.12 |
| soft capsule | base | solution | |

### Example 2. Soft Capsule Containing Aqueous Solution and Fat-Soluble Suspension

Soft capsules containing an aqueous solution and a fat-soluble suspension according to ingredients and contents shown in Table 2 below were prepared. In detail, purified water and potassium hydroxide were put into a stainless container, and dissolved therein. Polyethylene glycol 600, butylhydroxytoluene, and povidone were put into a separate drug injection tank and dissolved by raising the temperature to 65° C. to prepare a water-soluble base (first base). Next, the raising of the temperature was stopped, and ibuprofen among the first active ingredients and potassium hydroxide aqueous solution were put into a drug injection tank and dissolved. Then, the temperature of the drug injection tank was set to 65° C., and thus when the temperature of the contents reached 65° C., pamabrom was added and dissolved. Afterwards, through filtration and defoaming processes, a filling composition for a water-soluble soft capsule (first composition) was prepared.

Soybean oil, hydrogenated coconut oil, and white lead were put into a drug injection tank and dissolved by raising the temperature to 55° C., to prepare a fat-soluble base (second base). Then, magnesium oxide and caffeine anhydride as second active ingredients were added to a drug injection tank, stirred, and subjected to milling, filtration, and defoaming processes to prepare a filling composition for a fat-soluble soft capsule (second composition). Then, by using a soft capsule-gelatin shell formulation manufacturing device, gelatin and sorbitol sorbitan solution were used to prepare an outer shell of a soft capsule. These two contents were then injected and sealed by using a multi(double)-fill soft capsule molding device.

TABLE 2

| | Blending | | |
|---|---|---|---|
| Division | purpose | Ingredient | Content (wt %) |
| Filling | First active | Ibuprofen | 19.42 |
| composition for | ingredient | Pamabrom | 2.43 |
| water-soluble | Additive | Polyethylene glycol600 | 20.85 |
| soft | | Purified water | 2.43 |
| capsule | | Potassium hydroxide | 2.43 |
| | | Povidone | 0.97 |
| | | Butylhydroxytoluene | 0.02 |
| Filling | Second active | Magnesium oxide | 4.85 |
| composition for | ingredient | Caffein anhydrous | 3.88 |
| fat-soluble | Additive | Soybean oil | 7.96 |
| soft | | Hydrogenated | 1.75 |
| capsule | | coconut oil | |
| | | White lead | 0.58 |
| | | Lecithin | 0.39 |
| Gelatin shell | Gelatin shell | Succinic acid gelatin | 20.35 |
| formulation of soft | base | Sorbitol sorbitan | 11.68 |
| capsule | | solution | |

### Example 3. Soft Capsule Containing Aqueous Solution and Fat-Soluble Solution

Soft capsules containing an aqueous solution and a fat-soluble solution according to ingredients and contents shown in Table 3 below were prepared. In detail, polyethylene glycol as a water-soluble base was mixed with propylene glycol as a dissolution auxiliary agent, and the mixture was mixed at a speed of 150 rpm for 10 minutes. Then, rosuvastatin was slowly added thereto at 37° C., and until completely dissolved, the mixture was allowed for dissolution while mixing at a speed of about 400 rpm. Next, the resulting mixture was filtered through 200 mesh and cooled, and air bubbles were removed therefrom to prepare a filling composition for a water-soluble soft capsule (first composition).

After flowing nitrogen gas into a separate container, omega-3-acid ethyl ester 90 was added thereto and filtered through 200 mesh. Nitrogen gas was then injected and sealed to prepare a filling composition for a fat-soluble soft capsule (second composition).

These two types of filling compositions for a soft capsule thus prepared were mixed with gelatin as a soft capsule base and sorbitol sorbitan solution and water as plasticizers to form gel mass. Then, a soft capsule was prepared by using a multi(double)-fill shaping device using the gel mass and a soft capsule gelatin shell formulation formed in a thin and wide ribbon form.

TABLE 3

| | Blending | | |
|---|---|---|---|
| Division | purpose | Ingredient | Content (wt %) |
| Filling composition | First active | Rosuvastatincalcium | 0.35 |
| in water-soluble | ingredient | | |
| soft capsule | Additive | Polyethylene glycol | 2.84 |
| | | Propylene glycol | 2.79 |
| Filling composition | Second | Omega-3-acid | 66.44 |
| in fat-soluble soft | active | ethyl ester 90 | |
| capsule | ingredient | | |
| Gelatin shell | Gelatin shell | Gelatin | 17.52 |
| formulation of soft | base | Sorbitol | 10.06 |
| capsule | | sorbitan solution | |

### Example 4

Soft capsules containing an aqueous solution and a fat-soluble solution according to ingredients and contents shown in Table 4 below were prepared. In detail, purified water and potassium hydroxide were put into a container, and dissolved therein. Polyethylene glycol 600, butylhydroxytoluene, and povidone were put into a separate drug injection tank and dissolved by raising the temperature to 65° C. to prepare a water-soluble base (first base). Next, the raising of the temperature was stopped, and biotin and folic acid among the first active ingredients and potassium hydroxide aqueous solution were put into a drug injection tank and dissolved therein. Then, the temperature of the drug injection tank was set to 65° C., and thus when the temperature of the contents reached 65° C., the first active ingredients were added one by one in turn and dissolved. Afterwards, through filtration and defoaming processes, a filling composition for a water-soluble soft capsule (first composition) was prepared.

Separately, a liquid second active ingredient was added to a drug injection tank, stirred, and then subjected to filtration and defoaming processes to prepare a filling composition for a fat-soluble soft capsule (second composition). Then, by using a soft capsule-gelatin shell formulation manufacturing device, gelatin and sorbitol sorbitan solution were used to prepare an outer shell of a soft capsule. These two contents were then injected and sealed by using a multi(double)-fill soft capsule molding device.

TABLE 4

| | Blending | | |
|---|---|---|---|
| Division | purpose | Ingredient | Content (wt %) |
| Filling | First active | Thiamine nitrate | 2.16 |
| composition in | ingredient | Riboflavin | 1.03 |
| water-soluble | | Nicotinic acid amide | 1.72 |
| soft | | Calcium pantothenate | 1.72 |
| capsule | | Pyridoxine hydrochloride | 2.16 |
| | | Biotin | 0.01 |
| | | Follic acid | 0.02 |
| | | Cyanocobalamin | 0.0002 |
| | | Ascorbic acid | 2.16 |
| | Additive | Polyethylene glycol600 | 14.64 |
| | | Purified water | 1.81 |
| | | Propylene glycol | 1.21 |
| | | Povidone | 1.51 |
| | | Butylhydroxytoluene | 0.03 |
| | | Potassium hydroxide | 0.002 |
| | | Colorant | 0.00 |
| Filling | Second | Retinol palmitate | 0.05 |
| composition in | active | Cholecalciferol concentrate | 0.01 |
| fat-soluble | ingredient | Tocopherol acetate | 4.31 |
| soft | | Gamma-oryzanol | 0.43 |
| capsule | | Phytonadione | 0.004 |
| | Additive | Soybean oil | 33.99 |
| Gelatin shell | Gelatin shell | Gelatin | 19.72 |
| formulation of soft | base | Sorbitol | 11.32 |
| capsule | | sorbitan solution | |

As a result, it was confirmed that, in the capsule formulation of Example 4, the first liquid phase and the second liquid phase did not mix with each other and were present as separate phases due to the water-soluble or fat-soluble properties of each base without an additional surfactant or the presence of a physical layer.

### Example 5. Soft Capsule Containing Fat-Soluble Suspension and Fat-Soluble Suspension

Soft capsules containing a fat-soluble suspension and a fat-soluble suspension according to ingredients and contents shown in Table 5 below were prepared. In detail, yellow lead as a suspending agent was added to soybean oil as an excipient, and the mixture was mixed at about 55° C. at a speed of 600 rpm until the ingredients added were completely dissolved. Then, lecithin as a wetting agent and a first active ingredient were added thereto and mixed for about 15 minutes at the same speed until being blended homogeneously. Afterwards, the blended mixture was milled by using a colloid mill, filtered through 80 mesh, and cooled, and air bubbles were removed therefrom to prepare a fat-soluble suspension (first composition). In the same manner as described above, a second active ingredient was added thereto to prepare a fat-soluble suspension (second composition), and by using a multi(double)-fill soft capsule molding device in the same manner as in Example 1, the two types of contents were then injected and sealed.

TABLE 5

| | Blending | | |
|---|---|---|---|
| Division | purpose | Ingredient | Content (wt %) |
| Filling | First active | Tocopherol acetate | 30.94 |
| composition | ingredient | Magnesium oxide | 15.48 |
| in fat-soluble | Additive | Soybean oil | 10.00 |
| soft capsule | | Yellow lead | 0.40 |
| | | Lecithin | 0.56 |
| Filling | Second | Pyridoxine hydrochloride | 2.48 |
| composition | active | Thiamine nitrate | 1.86 |
| in fat-soluble | ingredient | Benfothiamine | 1.24 |
| soft capsule | | Nicotinic acid amide | 0.62 |
| | | Gamma-oryzanol | 0.31 |
| | Additive | Soybean oil | 9.31 |
| | | Yellow lead | 0.85 |
| | | Lecithin | 0.25 |
| Gelatin shell | Gelatin shell | Gelatin | 17.37 |
| formulation | base | Concentrated | 8.33 |
| of soft | | glycerin | |
| capsule | | | |

### Comparative Examples

### Comparative Example 1. Soft Capsule Containing Aqueous Solution and Fat-Soluble Suspension

Soft capsules containing an aqueous solution and a fat-soluble suspension according to ingredients and contents shown in Table 1 above were prepared. In detail, a soft capsule was prepared in the same manner as in Example 1, except that a shaping device (by YUILPHARM TECH) was used after mixing a filling composition for a water-soluble soft capsule and a filling composition for a fat-soluble soft capsule by using a homogenizer at a speed of 150 rpm for about 10 minutes.

### Comparative Example 2. Soft Capsule Containing Water-Soluble Suspension

Soft capsules containing an aqueous suspension according to ingredients and contents shown in Table 6 below were prepared. In detail, polyethylene glycol 400 and concentrated glycerin were added to a drug injection tank and mixed, and polyethylene glycol 4000 and butylhydroxytoluene were dissolved sequentially while raising the temperature to 60° C. Then, a first active ingredient and a second active ingredient were added and mixed, and the mixture was subjected to filtration and defoaming processes to prepare a water-soluble suspension.

Then, by using a soft capsule-gelatin shell formulation manufacturing device, gelatin and sorbitol sorbitan solution were used to prepare an outer shell of a soft capsule. After injecting these contents by using a typical soft capsule molding device, the capsules were sealed.

TABLE 6

| | Blending | | |
|---|---|---|---|
| Division | purpose | Ingredient | Content (wt %) |
| Filling | First | Acetaminophen | 16.67 |
| composition | active | Guaifenesin | 1.93 |
| in | ingredient | Pseudoephedrinehydro- | 1.39 |
| water-soluble | | chloride | |
| soft capsule | | DL-methylephedrine HCl | 1.16 |
| | | Triprolidine | 0.06 |
| | | hydrochloridehydrate | |
| | Second | Ascorbic acid | 7.71 |
| | active | Riboflavin | 0.19 |
| | ingredient | Thiamine nitrate | 0.39 |
| | Additive | Polyethylene glycol 400 | 40.87 |
| | | Polyethylene glycol 4000 | 1.45 |
| | | Concentrated glycerin | 2.22 |
| | | Butylhydroxytoluene | 0.04 |
| Gelatin shell | Gelatin shell | Gelatin | 16.47 |
| formulation of soft | base | Sorbitol | 9.46 |
| capsule | | sorbitan solution | |

### Comparative Example 3. Soft Capsule Containing Fat-Soluble Suspension

By using ingredients and contents shown in Table 7 below, yellow lead and hard fat as suspending agents were added to soybean oil as an excipient, and mixed at a speed of 600 rpm at about 55° C. until the ingredients added therein were completely dissolved. Then, lecithin as a wetting agent and first and second active ingredients were added thereto and mixed at the sample speed for about 15 minutes until being blended homogeneously. Afterwards, the blended mixture was milled by using a colloid mill, filtered through 80 mesh, and cooled, and air bubbles were removed therefrom to prepare a fat-soluble suspension (first composition). Then, by using a soft capsule-gelatin shell formulation manufacturing device, gelatin and non-crystal sorbitol solution were used to prepare an outer shell of a soft capsule. These contents were then injected and sealed by using a typical soft capsule molding device.

TABLE 7

| | Blending | | |
|---|---|---|---|
| Division | purpose | Ingredient | Content (wt %) |
| Filling | First | Acetaminophen | 18.56 |
| composition | active | Guaifenesin | 2.15 |
| in | ingredient | Pseudoephedrinehydro- | 1.55 |
| fat-soluble | | chloride | |
| soft capsule | | DL-methylephedrine HCl | 1.29 |
| | | Triprolidine | 0.07 |
| | | hydrochloridehydrate | |
| | Second | Ascorbic acid | 8.59 |
| | active | Riboflavin | 0.21 |
| | ingredient | Thiamine nitrate | 0.43 |
| | Additive | Soybean oil | 31.26 |
| | | Hard fat | 4.41 |
| | | Yellow lead | 1.47 |
| | | Lecithin | 1.13 |
| | | Tocopherol acetate | 0.03 |
| Gelatin shell | Gelatin | Gelatin | 17.24 |
| formulation | shell | Concentrated glycerin | 5.40 |
| of soft | base | Amorphous sorbitol | 6.23 |
| capsule | | solution | |

### Comparative Example 4. Soft Capsule Containing Aqueous Solution

By using ingredients and contents shown in Table 8 below, a filling composition for a water-soluble soft capsule (first composition) was prepared in the same manner as the water-soluble solution of Example 2. Then, by using a soft capsule-gelatin shell formulation manufacturing device, gelatin and sorbitol sorbitan solution were used to prepare an outer shell of a soft capsule. Then, these contents were then injected and sealed by using a typical soft capsule molding device.

TABLE 8

| | Blending | | |
|---|---|---|---|
| Division | purpose | Ingredient | Content (wt %) |
| Filling | First active | Ibuprofen | 28.57 |
| composition | ingredient | Pamabrom | 3.57 |
| with | Additive | Polyethylene glycol600 | 30.68 |
| aqueous | | Purified water | 3.57 |
| solution | | Potassium hydroxide | 3.57 |
| | | Povidone | 1.43 |
| | | Butylhydroxytoluene | 0.04 |
| Gelatin shell | Gelatin | Succinic acid gelatin | 18.15 |
| formulation of soft | shell | Sorbitol | 10.42 |
| capsule | base | sorbitan solution | |

### Comparative Example 5. Soft Capsule Containing Water-Soluble Suspension

By using ingredients and contents shown in Table 9 below, a filling composition for a water-soluble soft capsule (second composition) was prepared in the same manner as in Example 2.

TABLE 9

| | Blending | | |
|---|---|---|---|
| Division | purpose | Ingredient | Content (wt %) |
| Filling composition | First active | Ibuprofen | 28.57 |
| in water-soluble | ingredient | Pamabrom | 3.57 |
| suspension | Second active | Magnesium oxide | 7.14 |
| | ingredient | Caffein anhydrous | 5.71 |
| | Additive | Polyethylene glycol600 | 49.50 |
| | | Polyethylene glycol 4000 | 5.00 |
| | | Polysorbate 80 | 0.50 |

As a result, due to a reaction between polyethylene glycol 600 and magnesium oxide, these are determined to be unsuitable as a filling composition, and thus a soft capsule was not prepared.

### Comparative Example 6. Soft Capsule Containing Fat-Soluble Suspension

By using ingredients and contents shown in Table 10 below, a filling composition for a fat-soluble soft capsule was prepared in the same manner as in Comparative Example 3. Then, by using a soft capsule-gelatin shell formulation manufacturing device, gelatin and sorbitol sorbitan solution were used to prepare an outer shell of a soft capsule. After injecting these contents by using a typical soft capsule molding device, the capsules were sealed.

TABLE 10

| | Blending | | |
|---|---|---|---|
| Division | purpose | Ingredient | Content (wt %) |
| Filling | First active | Ibuprofen | 19.42 |
| composition in | ingredient | Pamabrom | 2.43 |
| fat-soluble | Second | Magnesium | 4.85 |
| soft | active | oxide | |
| capsule | ingredient | Caffein anhydrous | 3.88 |
| | Additive | Soybean oil | 30.58 |
| | | Hydrogenated | 4.08 |
| | | coconut oil | |
| | | White lead | 1.36 |
| | | Lecithin | 1.36 |
| Gelatin | Gelatin | Succinic acid | 20.35 |
| shell | shell | gelatin | |
| formulation | base | Sorbitol | 11.68 |
| of soft capsule | | sorbitan solution | |

### Comparative Example 7. Soft Capsule Containing Fat-Soluble Suspension

By using ingredients and contents shown in Table 11 below, a filling composition for a fat-soluble soft capsule was prepared in the same manner as in Comparative Example 3.

Then, by using a soft capsule-gelatin shell formulation manufacturing device, gelatin and concentrated glycerin were used to prepare an outer shell of a soft capsule. These contents were then injected and sealed by using a typical soft capsule molding device.

TABLE 11

| Division | Ingredient | Content (wt %) |
|---|---|---|
| Active | Tocopherol acetate | 30.94 |
| ingredient | Magnesium oxide | 15.48 |
| | Pyridoxine hydrochloride | 2.48 |
| | Thiamine nitrate | 1.86 |
| | Benfothiamine | 1.24 |
| | Nicotinic acid amide | 0.62 |
| | Gamma-oryzanol | 0.31 |
| Additive | Soybean oil | 17.28 |
| | Yellow lead | 2.23 |
| | Lecithin | 1.49 |
| | Polysorbate 80 | 0.37 |
| Gelatin shell | Gelatin | 17.37 |
| formulation of soft | Concentrated | 8.33 |
| capsule | glycerin | |

### Experimental Examples

### Experimental Example 1. Evaluation of Content Stability of Active Ingredients

For the soft capsules of Example 1 and Comparative Example 2, the content stability of the active ingredients over time was evaluated. In detail, the composition of each of Example 1 and Comparative Example 2 was PTP-wrapped with PVC film and A1-foil, and the content of the active ingredients were measured at 40° C. and 75 RH % at 2-week intervals for 1 month. The content test was carried out by high-speed liquid chromatography according to the content test method of each active ingredient set by COSMAX Pharma, and the results are shown in Tables 12 and 13 below.

TABLE 1
Example 1

Elapsed period (unit: week)

| Ingredient | 0 | 2 | 4 |
|---|---|---|---|
| Acetaminophen | 97.1% | 97.0 | 96.4 |
| Guaifenesin | 96.8% | 96.8 | 97.2 |
| Pseudoephedrinehydrochloride | 96.4% | 96.2 | 97.7 |
| DL-methylephedrine HCl | 100.5% | 100.1 | 100.7 |
| Triprolidine | 105.8% | 104.8 | 104.8 |
| hydrochloridehydrate | | | |
| Ascorbic acid | 98.8% | 98.7 | 98.7 |
| Riboflavin | 99.0% | 97.2 | 95.8 |
| Thiamine nitrate | 101.1% | 96.5 | 93.2 |

TABLE 13
Comparative Example 2

Elapsed period (unit: week)

| Ingredient | 0 | 2 | 4 |
|---|---|---|---|
| Acetaminophen | 97.9 | 97.8 | 98.0 |
| Guaifenesin | 98.2 | 99.0 | 97.0 |
| Pseudoephedrinehydrochloride | 99.4 | 98.9 | 98.1 |
| DL-methylephedrine HCl | 98.5 | 98.7 | 97.4 |
| Triprolidine | 103.9 | 103.7 | 100.2 |
| hydrochloridehydrate | | | |
| Ascorbic acid | 93.8 | 88.5 | 77.4 |
| Riboflavin | 95.0 | 87.8 | 72.3 |
| Thiamine nitrate | 91.5 | 74.4 | 64.2 |

As a result, as shown in Tables 12 and 13, it was confirmed that the content stability of Example 1 was remarkably superior to Comparative Example 2. In detail, as the water-soluble active ingredients, i.e., water-soluble vitamins (e.g., ascorbic acid, riboflavin, and thiamin nitrate), contained in the water-soluble substance of Comparative Example 2 migrated to the gelatin shell formulation, the initial content of the active ingredients was detected low, and the content stability was confirmed to be significantly degraded over time. Meanwhile, in the case of Example 1, the reactivity between the active ingredients was minimized due to the phase separation, and thus the active ingredients were confirmed to have excellent content stability.

### Experimental Example 2. Dissolution Rate Evaluation 1

For each of the active ingredients of Example 1, Comparative Example 3, and Phenssac-Col soft capsule (by Jeil Pharmaceutical Co., Ltd.), i.e., acetaminophen, guaifenesin, pseudoephedrinehydrochloride, DL-methylephedrine HCl, and triprolidine hydrochloridehydrate, the dissolution rate was evaluation. In detail, a dissolution rate tendency was evaluated for each active ingredient up to 60 minutes from the start of the dissolution test at 50 rpm according to the dissolution test method II of the Korean Pharmacopoeia using 900 mL of water as a test solution, and the results are shown in Tables 14 to 18 below.

TABLE 14

| | Average dissolution rate (%, average ± | | | | | |
|---|---|---|---|---|---|---|
| | standard deviation) of acetaminophen | | | | | |
| Test group | 5 min. | 10 min. | 15 min. | 30 min. | 45 min. | 60 min. |
| Example 1 | 1.4 | 5.4 | 50.1 | 95.0 | 97.5 | 98.0 |
| Comparative | 0.0 | 1.4 | 6.6 | 30.5 | 53.1 | 68.2 |
| Example 3 | | | | | | |
| Phenssac-Col | 1.4 | 14.2 | 75.6 | 96.3 | 98.4 | 98.6 |
| Soft Cap. | | | | | | |

TABLE 15

| | Average dissolution rate (%, average ± | | | | | |
|---|---|---|---|---|---|---|
| | standard deviation) of guaifenesin | | | | | |
| Test group | 5 min. | 10 min. | 15 min. | 30 min. | 45 min. | 60 min. |
| Example 1 | 1.1 | 5.1 | 48.6 | 93.6 | 96.1 | 96.4 |
| Comparative | 0.0 | 2.7 | 10.0 | 37.1 | 61.5 | 76.8 |
| Example 3 | | | | | | |
| Phenssac-Col | 1.2 | 13.9 | 74.3 | 95.3 | 97.4 | 97.4 |
| Soft Cap. | | | | | | |

TABLE 16

| | Average dissolution rate (%, average ± | | | | | |
|---|---|---|---|---|---|---|
| | standard deviation) of pseudoephedrine HCl | | | | | |
| Test group | 5 min. | 10 min. | 15 min. | 30 min. | 45 min. | 60 min. |
| Example 1 | 0.0 | 3.5 | 47.6 | 93.6 | 96.4 | 98.3 |
| Comparative | 0.0 | 0.0 | 10.8 | 39.7 | 65.6 | 81.6 |
| Example 3 | | | | | | |
| Phenssac-Col | 0.0 | 12.5 | 70.2 | 92.9 | 96.7 | 97.5 |
| Soft Cap. | | | | | | |

TABLE 17

| | Average dissolution rate (%, average ± | | | | | |
|---|---|---|---|---|---|---|
| | standard deviation) of DL-methylephedrine HCl | | | | | |
| Test group | 5 min. | 10 min. | 15 min. | 30 min. | 45 min. | 60 min. |
| Example 1 | 0.0 | 3.1 | 46.3 | 92.6 | 95.5 | 96.9 |
| Comparative | 0.0 | 0.0 | 10.6 | 39.6 | 66.0 | 82.5 |
| Example 3 | | | | | | |
| Phenssac-Col | 0.0 | 11.8 | 70.2 | 92.8 | 96.7 | 97.7 |
| Soft Cap. | | | | | | |

TABLE 18

| | Average dissolution rate (%, average ± | | | | | |
|---|---|---|---|---|---|---|
| | standard deviation) of triprolidine hydrochloride | | | | | |
| Test group | 5 min. | 10 min. | 15 min. | 30 min. | 45 min. | 60 min. |
| Example 1 | 10.1 | 14.5 | 43.7 | 83.2 | 83.2 | 83.7 |
| Comparative | 3.9 | 14.5 | 16.3 | 31.6 | 53.6 | 68.4 |
| Example | | | | | | |
| Phenssac-Col | 14.4 | 13.9 | 57.1 | 79.8 | 82.0 | 84.5 |
| Soft Cap. | | | | | | |

FIGS. 6 to 10 are each a graph comparing the average dissolution rates of acetaminophen, guaifenesin, pseudoephedrinehydrochloride, methylephedrine HCl, and triprolidine hydrochloride of Example 1, Comparative Example 3, and Phenssac-Col soft capsule.

As shown in FIGS. 6 to 10 and Tables 14 to 18, it was confirmed that the average dissolution rates for the active ingredients of Example 1 and all active ingredients of Phenssac-Col consisting of a composition similar to Example 1 reached 75% (30 minutes) or more. Meanwhile, in the case of Comparative Example 3, it showed a dissolution rate of less than half of Example 1 and Phenssac-Col soft capsule, and thus it was accordingly confirmed that the formulation containing the corresponding active ingredients was not suitable for the dissolution rate criterion. That is, even though the water-soluble liquid and the fat-soluble suspension are mixed in the capsule of the soft capsule according to an aspect, the same dissolution as the water-soluble liquid formulation was confirmed to be exhibited. Therefore, the filling composition for a soft capsule according to an aspect can be prescription designed based on a base having properties suitable for the characteristics and stability of each active ingredient without limitation in base selection, and even though two compositions with different properties coexist in the capsule, a soft capsule with improved pharmaceutical stability can be prepared by minimizing the reactivity between these two compositions.

### Experimental Example 3. Dissolution Rate Evaluation 2

The dissolution rates of ibuprofen and pamabrom, which are active ingredients of Example 2 and Comparative Examples 4 and 6, were evaluated in the same manner as in Experimental Example 2, and the results are shown in Tables 19 and 20 below.

TABLE 19

| | Average dissolution rate (%, average ± | | | | | |
|---|---|---|---|---|---|---|
| | standard deviation) of ibuprofen | | | | | |
| Test group | 5 min. | 10 min. | 15 min. | 30 min. | 45 min. | 60 min. |
| Example 2 | 0.29 | 8.34 | 57.87 | 87.32 | 92.66 | 95.53 |
| Comparative | 0.00 | 12.21 | 56.91 | 90.42 | 94.72 | 98.81 |
| Example 4 | | | | | | |
| Comparative | 0.00 | 2.91 | 5.78 | 9.85 | 10.12 | 10.34 |
| Example 6 | | | | | | |

TABLE 20

| | Average dissolution rate (%, average ± | | | | | |
|---|---|---|---|---|---|---|
| | standard deviation) of pamabrom | | | | | |
| Test group | 5 min. | 10 min. | 15 min. | 30 min. | 45 min. | 60 min. |
| Example 2 | 0.47 | 10.80 | 63.06 | 94.82 | 100.70 | 102.72 |
| Comparative | 0.00 | 14.74 | 64.77 | 95.17 | 101.29 | 102.09 |
| Example 4 | | | | | | |
| Comparative | 0.03 | 2.16 | 2.46 | 5.39 | 5.90 | 6.07 |
| Example 6 | | | | | | |

FIGS. 11 and 12 are graphs comparing the average dissolution rates of the active ingredients (ibuprofen and pamabrom) of Example 2 and Comparative Examples 4 and 6.

Referring to FIGS. 11 and 12 and Tables 19 and 20, Example 2 was confirmed to exhibit the average dissolution rate similar to that of Comparative Example 4 (existing liquid composition). Meanwhile, in the case of Comparative Example 6, the significantly lower average dissolution rate than Example 2 was exhibited, confirming that no dissolution occurred.

That is, despite containing different compositions in the capsule, the soft capsule according to an aspect was able to exhibit an effect equivalent to that of an existing liquid composition.

### Experimental Example 4. Accelerated Stability Test

### 4-1. Content Test

The samples prepared in Example 2 and Comparative Example 4 were stored under conditions of 40±2° C. and 75±5% RH. Then, after 0, 2, and 4 weeks, the contents of the main components were calculated for each sample, and the results are shown in Tables 21 and 22 below.

TABLE 21
Example 2

| | Elapsed period (unit: week) | | |
|---|---|---|---|
| Ingredient | 0 | 2 | 4 |
| Ibuprofen | 100.01% | 99.93% | 99.76% |
| Pamabrom | 99.95% | 99.87% | 99.81% |
| Caffein anhydrous | 99.82% | 99.78% | 99.72% |
| Magnesium oxide | 100.03% | 99.92% | 99.84% |

TABLE 22
Comparative Example 4

| | Elapsed period (unit: week) | | |
|---|---|---|---|
| Ingredient | 0 | 2 | 4 |
| Ibuprofen | 100.05% | 99.99% | 99.92% |
| Pamabrom | 100.03% | 99.97% | 99.94% |

As a result, as shown in Tables 21 and 22, both Examples 2 and Comparative Example 4 showed little change in the contents even during the storage period of 4 weeks. Therefore, the soft capsule according to an aspect has excellent formulation stability, and thus can be stored for a long period of time.

### 4-2. Dissolution Test

After the soft capsules of Example 2 and Comparative Example 4 were stored for the same period of time under the same conditions as 4-1, the dissolution of the main components was calculated according to Equation 1 below, and the results are shown in Tables 23 and 24.

$$Dissolution rate ( \% ) = \frac{Area ratio of test liquid * standard quantity * dilution factor of test liquid * standard purity}{Stanard area ratio of standard liquid * number of samples * allowed quantity * dilution factor of standard liquid} * 100 \qquad [ Equation 1 ]$$

TABLE 23
Example 2

| | Elapsed period (unit: week) | | |
|---|---|---|---|
| Ingredient | 0 | 2 | 4 |
| Ibuprofen | 91.64% | 87.32% | 87.39% |
| Pamabrom | 93.28% | 94.82% | 91.25% |

TABLE 24
Comparative Example 4

| | Elapsed period (unit: week) | | |
|---|---|---|---|
| Ingredient | 0 | 2 | 4 |
| Ibuprofen | 90.42% | 88.16% | 87.95% |
| Pamabrom | 95.17% | 94.12% | 92.67 |

As a result, as shown in Tables 23 and 24, both Examples 2 and Comparative Example 4 showed little change in the dissolution even during the storage period of 4 weeks.

### 4-3. Test on Impurities

After the soft capsules of Example 2 and Comparative Example 4 were stored for the same period of time under the same conditions as 4-1, the impurities of the main components was calculated according to Equations 2 to 4 (for ibuprofen) and 5 (for pamabrom) below, and the results are shown in Tables 25 and 26.

$$Impurity B ( \% ) \frac{Peak area of impurity B in test liquid}{Peak area of impurity B in standard liquid ( 2 )} \qquad [ Equation 2 ]$$

$$I ndividual impurities ( \% ) = \frac{Peak areas of those other than main ingredients in test liquid}{Peak area of main ingredients in standard liquid ( 1 )} \qquad [ Equation 3 ]$$

$$Total impurites ( \% ) = \frac{Sum of each peak area in test liquid}{Peak area of main ingredients in standard liquid ( 1 )} \qquad [ Equation 4 ]$$

$$Theophyline content ( \% ) = \frac{Area ratio of test liquid * standard quantity * dilution factor of test liquid * standard purity}{standard area ratio of standard liquid * number of samples * allowed quantity * dilution factor of standard liquid} \times 100 \qquad [ Equation 5 ]$$

TABLE 25
Example 2

| | | Elapsed period (unit: week) | | |
|---|---|---|---|---|
| Ingredient | | 0 | 2 | 4 |
| Ibuprofen | 1) Impurity B | 0.02% | 0.02% | 0.02% |
| | (0.3% or less) | | | |
| | 2) Individual | 0.00% | 0.00% | 0.00% |
| | impurity | | | |
| | (0.3% or less) | | | |
| | 3) Total impurities | 0.00% | 0.00% | 0.00% |
| | (0.7% or less) | | | |
| Pamabrom | Theophyline | 0.02% | 0.04% | 0.04% |
| | (0.5% or less) | | | |

TABLE 26
Comparative Example 4

| | | Elapsed period (unit: week) | | |
|---|---|---|---|---|
| Ingredient | | 0 | 2 | 4 |
| Ibuprofen | 1) Impurity B | 0.03% | 0.03% | 0.03% |
| | (0.3% or less) | | | |
| | 2) Individual | 0.00% | 0.00% | 0.00% |
| | impurity | | | |
| | (0.3% or less) | | | |
| | 3) Total impurities | 0.00% | 0.00% | 0.00% |
| | (0.7% or less) | | | |
| Pamabrom | Theophyline | 0.03% | 0.04% | 0.04% |
| | (0.5% or less) | | | |

As a result, as shown in Tables 25 and 26, both Examples 2 and Comparative Example 4 showed little change in the contents of impurities even during the storage period of 4 weeks.

That is, the filling composition for a soft capsule according to an aspect does not cause a reaction among the active ingredients so that it can be filled in one soft capsule without a need to prepare a separate capsule. The filling composition for a soft capsule according to an aspect also exhibit a dissolution rate and stability similar to those of existing soft capsules, and thus items containing various active ingredients can be easily developed.

### Experimental Example 5. Evaluation of Comparison with Existing Soft Capsules 5-1. Content Test

By measuring the contents of the soft capsule of Example 3 and Rosumega soft capsule (by Kuhnil Pharm. Co, Ltd.) (hereinafter, referred to as a control drug), the effects of the filling composition for a water-soluble soft capsule and the filling composition for a fat-soluble soft capsule on the contents of each active ingredient were evaluated. In detail, the content test on rosuvastatin calcium was carried out by high-speed liquid chromatography according to the anti-content test method of rosuvastatin calcium of the US Pharmacopeia. The content test on omega-3-acid ethyl ester 90 was carried out by high-speed liquid chromatography according to the composition of fatty acid in omega-3 acids (2.4.29) of European Pharmacopoeia, and the results are shown in Table 27 below.

TABLE 27

| | Content | | | |
|---|---|---|---|---|
| | | Omega-3-acid ethyl ester 90 (mg/g) | | |
| | | | | Total of |
| | | | | EPA and |
| | | EPA Ethyl | DHA Ethyl | DHA |
| | | ester | ester | 800.0 mg/g |
| | Rosuvastatin | 430 mg/g to | 347 mg/g to | to 882.0 |
| Test group | Calcium (%) | 495 mg/g | 403 mg/g | mg/g |
| Example 3 | 98 | 439.4 | 397.5 | 836.9 |
| Control drug | 101 | 437.4 | 396.0 | 833.5 |

As a result, as shown in Table 27, in the case of Example 3, EPA ethyl ester, DHA ethyl ester, and the sum of EPA and DHA in the items of the control drug of omega-3-acid ethyl ester 90 content test all showed similar values. In addition, the calcium content of rosuvastatin calcium was also 98%, indicating a result of suitable for the content standard.

### 5-2. Disintegration Evaluation

For the soft capsule of Example 3 and the control drug, the disintegration time was measured in water at 37° C. according to the disintegration test method of the general test method of the Korean Pharmacopoeia, and the results are shown in Table 28.

TABLE 28

| Test group | Disintegration time (min.) |
|---|---|
| Example 3 | 6 minutes 45 seconds |
| Control drug | 8 minutes 11 seconds |

As a result, as shown in Table 28, the soft capsule of Example 3 showed the disintegration time similar to that of the control drug. Therefore, it can be seen that the soft capsule according to an aspect did not affect the disintegration by minimizing transition between the filling composition for a water-soluble soft capsule and the filling composition for a fat-soluble soft capsule.

### 5-3. Dissolution Rate Evaluation

The dissolution rates of the soft capsule of Example 3 and the control drug were confirmed in the same manner as in Experimental Example 2, and analyzed according to the analysis conditions of the anti-dissolution test method TEST1 of the US Pharmacopoeia. The test was carried out until the average dissolution rate of Rosumega soft capsule reached 85% or more, and the dissolution trend was evaluated for each time period, and the results are shown in Table 29 below.

TABLE 29

Average dissolution rate
(%)(average ± standard deviation)

| Test group | 5 min. | 10 min. | 15 min. | 30 min. |
|---|---|---|---|---|
| Example 3 | 2.1 | 24.9 | 97.4 | 100.7 |
| Control drug | 37.9 | 71.1 | 86.6 | 95.1 |

FIG. 13 is a graph comparing the average dissolution rates of the active ingredient (rosuvastatin) of Example 3 and Rosumega soft capsule.

As shown in FIG. 13 and Table 29, it was confirmed that the average dissolution rates of the soft capsule of Example 3 and the control drug reached 85% or more (30 minutes). In the case of the control drug, the initial dissolution rate was higher than that of the soft capsule of Example 3 since the control drug was coated with rosuvastatin calcium, but the dissolution rate showed a gradual increase. Meanwhile, in the case of the soft capsule of Example 3, the initial dissolution rate was lower than that of the control drug due to the presence of a lag time, but the dissolution rate was rapidly increased after rupture, resulting in a final dissolution rate of 97.4% at 15 minutes, which is about 10% higher than that of the control drug. That is, it can be seen that the soft capsule according to an aspect shows the equivalence to the existing soft capsule, and even through two types of compositions having different efficacy and release characteristics coexist in the capsule, a soft capsule with improved pharmaceutical stability can be prepared by minimizing the reactivity.

### Experimental Example 6. Resolution Test

To prepare capsules of a suspension (fat-soluble-fat-soluble or water-soluble-water-soluble) having the same properties among capsule formulations according to an aspect, contents each content should be present in separate phases that do not mix with each other after being filled in a capsule. Therefore, the conditions for examining formulations of suspensions having the same properties were confirmed. In detail, after preparing a first fat-soluble suspension (Rx 1 to 4) and a second fat-soluble suspension (Rx 5 to 8) that having the same properties, the first fat-soluble suspension and the second fat-soluble suspension were allowed to stand for 24 hours. Then, it was confirmed with naked eyes whether layer separation had occurred. Afterwards, centrifugation was performed at 1,500 rpm for a total of 15 minutes while checking the condition at 5-minute intervals, and the results are shown in Tables 30 and 31.

TABLE 30

| Time | Rx1 | Rx2 | Rx3 | Rx4 |
|---|---|---|---|---|
| 5 min. | Separated | Not separated | Not separated | Not separated |
| 10 min. | — | Separated | Not separated | Not separated |
| 15 min. | — | — | Separated | Not separated |

TABLE 31

| Time | Rx5 | Rx6 | Rx7 | Rx8 |
|---|---|---|---|---|
| 5 min. | Separated | Not separated | Not separated | Not separated |
| 10 min. | — | Separated | Not separated | Not separated |
| 15 min. | — | — | Separated | Not separated |

As a result, as shown in Tables 30 and 31, the first fat-soluble suspension and the second fat-soluble suspension did not undergo phase separation after 5 minutes of the preparation, but the occurrence of phase separation was observed at 10 minutes (Rx 2 and 6) and 15 minutes (Rx 3 and 7). Based on these results, the first fat-soluble suspension and the second fat-soluble suspension, in which the layers were separated at the same time, were stored in the upper/lower layers in a round PET bottle, and then left for 2 weeks under conditions of 40±2° C. and 75±5% RH. Afterwards, the conditions of phase separation were checked, and the results are shown in Table 32.

TABLE 32

| | Sample 1 | Sample 2 | Sample 3 | Sample 4 |
|---|---|---|---|---|
| Period | (Rx1 + Rx5) | (Rx2 + Rx6) | (Rx3 + Rx7) | (Rx4 + Rx8) |
| 2 weeks | Miscible | Miscible | Miscible | Not miscible |

As a result, as shown in Table 32, in the case of Sample 4, it was confirmed that the two phases were not miscible with each other. Meanwhile, in the case of Samples 1 to 3, the two phases were miscible and the boundary between the suspensions could not be clearly identified. Therefore, when examining formulations of suspensions having the same properties, a prescription satisfying the condition that layer separation does not occur after centrifugation at 1,500 rpm for 15 minutes must be selected.

### Experimental Example 7. Evaluation of Content Stability of Active Ingredients

For the compositions of Example 1 and Comparative Example 7, the content stability of the active ingredients over time was evaluated. In detail, the compositions 1 and 2 of Example 5 were stored in the upper/lower layers in a round PET bottle in consideration of specific gravity, and the composition of Comparative Example 7 was also stored in a round PET bottle. Then, the contents of active ingredients were measured at 2-week intervals for 1 month under conditions of 40° C. and 75 RH %. The content test was carried out by high-speed liquid chromatography according to the content test method of each active ingredient set by COSMAX Pharma, and the results are shown in Table 33 below.

TABLE 33

| | Elapsed time (unit: week) | | |
|---|---|---|---|
| Test group | 0 | 2 | 4 |
| Example 5 | 122.1% | 101.3% | 91.9% |
| Comparative Example 7 | 124.2% | 92.4% | 82.1% |

As a result, as shown in Table 33, in the case of Comparative Example 7, the magnesium oxide directly affected the pH of the ingredients so that the pH increases over time. Accordingly, the content of benfotiamine was significantly reduced to 82.1% at the 4th week of acceleration. Meanwhile, in Example 5, a composition containing magnesium oxide and a composition not containing magnesium oxide were prepared separately, and then, the transition or reactivity between the two compositions was minimized to prevent pH increase. As a result, the content of benfotiamine was 91.9%, which was about 10% higher than Comparative Example 7, at the 4th week of acceleration, and thus it was confirmed that the stability was improved. These results refer that the soft capsule formulation according to an embodiment has excellent content stability by minimizing reactivity between active ingredients by phase separation.

FIG. 14 is a diagram schematically illustrating a portion of a capsule molding device according to an embodiment of the present disclosure, and FIG. 15 is a diagram for explaining an injection unit of FIG. 14.

Referring to FIGS. 14 and 15, the capsule molding device according to an embodiment of the present disclosure includes a first receiving chamber (not shown), a second receiving chamber (not shown), a sheet forming unit (not shown), capsule molding units 21 and 22, and an injection unit 10.

The first receiving chamber (not shown) may accommodate a first liquid phase or a first suspension containing a base in which a first pharmaceutical active ingredient is dissolved or dispersed.

The second receiving chamber (not shown) may accommodate a second liquid phase or a second suspension containing a base in which a second pharmaceutical active ingredient is dissolved or dispersed. Here, the second liquid or the second suspension may include ingredients different from those of the first liquid or the first suspension.

The sheet forming unit (not shown) may form a sheet using a gelatin shell formulation base solution.

The capsule molding units 21 and 22 may include a pair of die rolls arranged adjacent to the sheet forming unit (not shown) to receive a sheet S formed in the sheet forming unit (not shown) and to encapsulate the sheet S. A first groove 211 may be formed in a first die roll 21, and a second groove 222 corresponding to the first groove 211 may be formed in a second die roll 22. While the first die roll 21 and the second die roll 22 rotate, a space in which a capsule M is formed may be secured through the corresponding first groove 211 and the second groove.

The injection unit 10 may be arranged adjacent to the outer periphery of the pair of die rolls 21 and 22, and may include an injection segment for injecting the first liquid phase or first suspension supplied from the first receiving chamber (not shown) and the second liquid phase or second suspension supplied from the second receiving chamber (not shown) into the encapsulated sheet.

As shown in FIG. 15, the injection segment includes a first injection hole h1 connected to the first receiving chamber (not shown) and a second injection hole h2 connected to the second receiving chamber (not shown). Also, in the injection segment, first ejection holes h11 and h12 being in communication with the first injection hole h1 and second ejection holes h21 and h22 being in communication with the second injection hole h2 may be formed.

In other words, the injection segment may inject the first liquid phase or first suspension through the first ejection holes h11 and h12 into a capsule M, and may inject the second liquid phase or second suspension through the second ejection holes h21 and h22 into the capsule M. Here, through the first ejection holes h11 and h12 and the second ejection holes h21 and h22 that are spaced apart from each other, the first liquid phase or first suspension or the second liquid phase or second suspension may be separately injected into a predetermined position of the capsule.

As another embodiment, the capsule molding device may further include a third receiving chamber (not shown) for receiving a third liquid phase or third suspension including a base in which a third pharmaceutical active ingredient is dissolved or dispersed. In this case, in the injection segment, a third ejection hole h3 connected to the third receiving chamber (not shown) and third ejection holes h31 and h33 being communication with the third injection hole h3 may be further formed.

The capsule molding device having the aforementioned structure can manufacture one soft capsule by simultaneously injecting the first liquid phase or first suspension and the second liquid phase or second suspension that contain different ingredients. As shown in FIGS. 1 to 5, the soft capsule consists of: the continuous first phase loaded with the first pharmaceutical active ingredient; and the continuous second phase loaded with the second pharmaceutical active ingredient, wherein the continuous first phase and the continuous second phase may be separated without a separate surfactant or the presence of a physical layer.

The foregoing descriptions are only for illustrating the disclosure, and it will be apparent to a person having ordinary skill in the art to which the present invention pertains that the embodiments disclosed herein can be easily modified into other specific forms without changing the technical spirit or essential features. Therefore, it should be understood that Examples described herein are illustrative in all respects and are not limited.

1. A capsule formulation comprising:
a continuous first phase of a first liquid phase or a first suspension comprising a base in which a first pharmaceutical active ingredient is dissolved or dispersed; and
a continuous second phase of a second liquid phase or a second suspension comprising a base in which a second pharmaceutical active ingredient is dissolved or dispersed.

2. The capsule formulation of claim 1, wherein the first phase and the second phase are present as separate phases from each other.

3. The capsule formulation of claim 1, wherein the capsule formulation is selected from the group consisting of the following cases:
(1) the first liquid phase or the first suspension contains a water-soluble base, and the second liquid phase or the second suspension contains a fat-soluble base, or vice versa;
(2) the first liquid phase or the first suspension contains a fat-soluble base, and the second liquid phase or the second suspension contains a fat-soluble base; and
(3) the first suspension contains a water-soluble base, and the second suspension contains a water-soluble base.

4. The capsule formulation of claim 1, wherein the first phase and the second phase are present as continuous phases, and an existence region of the first phase and an existence region of the second phase do not overlap each other.

5. The capsule formulation of claim 1, wherein one of the continuous first phase and the continuous second phase is present as two or multiple continuous phases that are separated from each other through the other phase.

6. The capsule formulation of claim 1, wherein the first liquid phase or suspension and the second liquid phase or suspension are ejected from two ejection holes of two separate receiving chambers of a capsule molding device, to simultaneously fill the capsule formulation and to be formulated in one capsule.

7. The capsule formulation of claim 1, wherein the continuous first and second phases in the capsule formulation are present as separate phases without an additional surfactant or the presence of a physical layer.

8. The capsule formulation of claim 1, wherein the first liquid phase or the first suspension is not miscible with the second liquid phase or the second suspension, since ingredients of the base of the first liquid phase or the first suspension are different from ingredients of the base of the second liquid phase or the second suspension, and physical properties of the first liquid phase or the first suspension are different from physical properties of the second liquid phase or the second suspension.

9. The capsule formulation of claim 1, wherein the water-soluble base is one selected from the group consisting of polyethylene glycol, propylene glycol, polymethylacrylate, polyethylene oxide, saturated polyglycorized glyceride (Gelucire), glycerol monostearate, carbohydrate, cellulose, polyvinyl alcohol, polyacrylic acid, propylene carbonate, diethylene glycol monoethyl ether (Transcutol), triacetin, concentrated glycerin, glycerol monocaprylocaprate, tetraglycol, and a combination thereof.

10. The capsule formulation of claim 1, wherein the fat-soluble base is selected from the group consisting of plant oil, such as soybean oil, coconut oil, corn oil, sunflower seed oil, grapeseed oil, rice bran oil, sesame oil, perilla oil, palm oil, olive oil, castor oil, and polyoxyl hydrogenated castor oil, animal oil, such as squalane, refined fish oil, and the like, mineral oil, such as vaseline, liquid paraffin, paraffin, ozokerite, ceresin, microcrystalline wax, and the like, and medium chain fatty acid triglyceride.

11. The capsule formulation of claim 1, wherein the first pharmaceutical active ingredient is selected from the group consisting of a non-steroidal anti-inflammatory drug (NSAID), an influenza medicine ingredient, a vitamin, and a statin-based drug.

12. The capsule formulation of claim 1, wherein the second pharmaceutical active ingredient is selected from the group consisting of omega-3 fatty acid or alkyl ester thereof, an antacid, and a vitamin.

13. The capsule formulation of claim 1, wherein the first or second pharmaceutical active ingredient is selected from the group consisting of acetaminophen, tramadol hydrochloride, aceclofenac, naproxen, esomeprazole magnesium trihydrate, cetirizine hydrochloride, pseudoephedrine hydrochloride, ebastine, cilostazol, glimepiride, metformin hydrochloride, sitagliptin phosphate salt hydrate, galantamine hydrobromide, saxagliptin monohydrate, amlodipine besylate, valsartan, telmisartan, hydrochlorothiazide, olmesartan medoxomil, rosuvastatin calcium, naproxen sodium, sumatriptan, pramipexole dihydrochloride monohydrate, clonidine HCl, nicardipine HCl, doxazosin mesylate, indapamide, felodipine, tolterodine l-tartrate, ritodrine HCl, tamsurosin HCl, nifedipine, isosorbide mononitrate, nisoldipine, venlafaxine HCl, trazodone HCl, paroxetine hydrochloride hydrate, roxatidine acetate HCl, metoclopramide hydrochloride hydrate, salbutamol sulphate, orphenadrine citrate, chlormadinone acetate, and oxybutynin hydrochloride.

14. The capsule formulation of claim 1, wherein the capsule formulation includes a pharmaceutical or nutracuetical gelatin shell base for a soft capsule.

15. The capsule formulation of claim 14, wherein the gelatin shell base for a soft capsule includes one or more ingredients selected from the group consisting of starch, arabic gum, tragacanth gum, karaya gum, ghatti gum, guar gum, locust bean gum, tara gum, konjac gum, algin, agar, carrageenan, pullulan, pectin, gellan, mannan, gelatin, xanthan gum, and a mixture thereof, and the plasticizer for a soft capsule includes one or more components selected from the group consisting of glycerin, ethyl phthalate, triethyl citrate, dibutyl sebacate, polyethylene glycol, triacetin, tributyl citrate, propylene glycol, and a mixture thereof.

16. A capsule molding device comprising: a first receiving chamber including a base, in which a first pharmaceutical active ingredient is dissolved or dispersed, and receiving a first liquid phase or a first suspension;
a second receiving chamber including a base, in which a second pharmaceutical active ingredient is dissolved or dispersed, and receiving a second liquid phase or a second suspension;
a sheet forming unit configured to form a sheet using a gelatin shell formulation base solution;
a capsule molding unit including a pair of die rolls arranged adjacent to the sheet forming unit to be supplied with a sheet formed in the sheet forming unit and being configured to encapsulate the sheet; and
an injection unit arranged adjacent to the outer periphery of the pair of die rolls and including an injection segment for injecting the first liquid phase or the first suspension supplied from the first receiving chamber and the second liquid phase or the second suspension supplied from the second receiving chamber into the encapsulated sheet, wherein
the first liquid phase or the first suspension and the second liquid phase or the second suspension are present as continuous phases that are separate from each other within the capsule formulation, and
the injection segment comprises: a first injection hole connected to the first receiving chamber; a second injection hole connected to the second receiving chamber; a first ejection hole being in communication with the first injection hole; and a second ejection hole being in communication with the second injection hole.

17. The capsule molding device of claim 16, further comprising at least one receiving chamber.
